(19) United States
(12) Patent Application Publication
(10) Pub. No.: US 2018/0185391 A1
(43) Pub. Date: Jul. 5, 2018

(54) Drug Treatment Of Psoriasis And Of Other Skin Disorders Associated With Inhibition Of Differentiation Of Epidermal Cells

(71) Applicants: Sinan Tas, Bor (TR); Oktay Avci, Izmir (TR)

(72) Inventors: Sinan Tas, Bor (TR); Oktay Avci, Izmir (TR)

(21) Appl. No.: 15/395,164

(22) Filed: Dec. 30, 2016

(51) Int. Cl. A61K 31/58; A61K 45/06; A61K 9/00

(52) U.S. Cl. CPC A61K 31/58; A61K 9/0014; A61K 45/06

(57) ABSTRACT

This invention concerns the use of cyclopamine, a naturally occurring steroidal alkaloid known for over thirty years, for the treatment of psoriasis and achievement of rapid clearance of the psoriatic skin lesions together with the reversion of the histopathological signs of disease to normalcy with no detectable side effects. The cyclopamine-induced clearance of psoriatic lesions from the skin of patients is associated with the causation of cellular differentiation in lesional epidermis and with the rapid disappearance of CD4(+) lymphocytes and other inflammatory cells from lesional skin. Skin lesions that show inhibition of differentiation of epidermal cells similar to that in psoriasis have been found to disappear following similar administrations of a selective inhibitor of Hedgehog/Smoothened signaling. Therapeutic compositions comprising of cyclopamine and a corticosteroid and/or the pre-treatment of lesions with a corticosteroid provide significantly further increased therapeutic effectiveness over the use of cyclopamine alone or a corticosteroid alone.

### CROSS REFERENCE

This application is a continuation-in-part of U.S. application Ser. No. 12/460,620, filed on 22 Jul. 2009 which is a continuation of U.S. application Ser. No. 10/682,662, filed on 9 Oct. 2003 which is a continuation-in-part of PCT/TR02/00017, filed on 19 Apr. 2001 designating the United States, and a continuation-in-part of PCT/TR01/00027, filed on 2 Jul. 2001 designating the United States. U.S. application Ser. No. 12/460,620, U.S. application Ser. No. 10/682,662, PCT/TR02/00017 and PCT/TR01/00027 are incorporated herein by reference in their entirety.

### BACKGROUND OF THE INVENTION

Psoriasis is a common chronic disease affecting around 2% of the general population and any person from infancy to old age. Its etiology and pathogenesis are unclear. Family and twin studies have suggested a polygenic influence but the nature and the mechanisms of action of the involved genes are unknown (Elder J T at al. (2001) Arch. Dermatol. 137:1447-1454). Environmental factors such as streptococcal infections and trauma to the skin are also associated with the formation of psoriatic lesions. How do these environmental factors contribute to psoriasis are again unclear. However the association with streptococcal infections, immunosuppressive actions of most of the current anti-psoriatic treatments and other findings are used widely to argue for an autoimmune nature of the disease and triggering by T Lymphocytes (Gottlieb S. L. et al (1995) Nat. Med. 1:442-447; Nickoloff B. J. (1999) Arch. Dermatol. 135:1104-1110; Krueger J. G. (2002) J. Am. Acad. Dermatol. 46:1-23).

Psoriasis vulgaris, characterized by well-demarcated scaly erythematous plaques of varying sizes anywhere on the skin, is the most common form of psoriasis.

Histopathological examinations of the psoriatic skin lesions reveal typical epidermal and dermal changes that include the following.

Epidermal hyperplasia with elongation and thickening of rete ridges.

Thinning of the suprapapillary epidermis.

Focal losses or decrease of the thickness of the granular layer of epidermis.

Infiltration of the subepidermal region of dermis with neutrophils and mononuclear inflammatory cells.

Dilatation and tortuosity of the capillaries in the papillary dermis, accompanied often by papillary edema.

“Munro microabcesses”, defined as focal intracorneal collections of neutrophils. Psoriatic skin lesions generally contain majority of the above listed histopathological changes and all changes can be found in a well-developed psoriatic lesion. In addition immunohistochemical and other indicators of the proliferating cells reveal presence of proliferating keratinocytes in the suprabasal layers of psoriatic lesional skin (proliferating cells are normally restricted to the basal layer of epidermis in healthy skin).

Obscurity of the etiology and pathogenesis of psoriasis has been reflected by the varied treatment strategies used for this disease (Spuls P. I. et al. (1997) Br. J. Dermatol. 137:943-949; Ashcroft D. M. et al. (2000) J. Clin. Pharm. Ther. 25:1-10; AI-Suwaidan S. N. et al. (2000) J. Am. Acad. Dermatol. 42:796-802; Lebwohl M. et al. (2001) J. Am. Acad. Dermatol. 45:487-498; Lebwohl M. et al. (2001) J. Am. Acad. Dermatol. 45:649-661). Currently common treatments include the topical corticosteroids, systemic administration of immunosuppressants (usually cyclosporine), ultraviolet irradiation of the affected skin with or without psoralen, systemic retinoids and systemic methothrexate (Spuls P. I. et al. (1997) Br. J. Dermatol. 137:943-949; Ashcroft D. M. et al. (2000) J. Clin. Pharm. Ther. 25:1-10; Lebwohl M. et al. (2001) J. Am. Acad. Dermatol. 45:487-498; Lebwohl M. et al. (2001) J. Am. Acad. Dermatol. 45:649-661). At present there is no cure for psoriasis and patients face a need for life-long treatment. Therefore relatively simpler treatments (usually topical keratolitics and corticosteroids) are considered first and when these fail, the more effective systemic treatments with more serious side effects are attempted. When the therapeutic aim is defined as the clearance of lesions, even the most effective systemic treatments are reported to fail in as many as one fourth of patients in large series (Spuls P. I. et al. (1997) Br. J. Dermatol. 137:943-949) and, because of the serious side effects, patients and physicians are advised that at present “complete clearance is not a realistic expectation” (AI-Suwaidan S. N. et al. (2000) J. Am. Acad. Dermatol. 42:796-802). In practice side effects usually limit the more potent treatments to shorter-term management [cyclosporine is nephrotoxic and strongly immunosuppressive, methotrexate is hepatotoxic, ultraviolet irradiation-psoralen is mutagenic/carcinogenic (Lebwohl M. et al. (2001) J. Am. Acad. Dermatol. 45:649-661)]. However, in the long term, topical carticosteroids are also not devoid of side effects (Lebwohl M. et al (2001) J. Am. Acad. Dermatol. 45:487-498). Currently available treatments require in general several weeks (typically 6-8 weeks) from the initiation of treatment to the appearance of objective clinical regression (AI-Suwaidan S. N. et al. (2000) J. Am. Acad. Dermatol. 42:796-802; Lebwohl M. et al. (2001) J. Am. Acad. Dermatol. 45:649-661).

Cyclopamine is a steroidal alkaloid that occurs naturally in the Veratrum plants. Teratogenicity of these plants on grazing pregnant animals led to the identification of cyclopamine as an active compound (Keeler R. F. (1969) Phytochemistry 8:223-225). How might have cyclopamine displayed teratogenicity was revealed by the finding that it is an inhibitor of the hedgehog/smoothened signal transduction pathway (Incardona J. P. et al. (1998) Development 125:3553-3562; Cooper M. K. et al. (1998) Science 280:1603-1607). The sonic hedgehog protein, a member of the hedgehog family of proteins, has been found to induce differentiation of its target cells, including the precursors of ventral cells in the developing central nervous system (Goodrich L. V. et al. (1998) Neuron 21:1243-1257). Inhibition of the hedgehog/smoothened pathway by cyclopamine in the developing chicken brain prevented formation of the ventral cells and caused holoprosencephaly (Incardona J. P. et al. (1998) Development 125:3553-3562; Cooper M. K. et al. (1998) Science 280:1603-1607), the common malformation observed in the lambs of the sheep grazing Veratrum (Binns W. et al. (1963) Am. J. Vet. Res. 24:11641175). Cyclopamine has been reported to inhibit cellular differentiation in other systems as well, including the differentiation of bone marrow cells to erythroid cells (Detmer K. et al. (2000) Dev. Biol. 222:242) and the differentiation of the urogenital sinus to prostate (Berman D. M. et al. (2000) J. Urol. 163:204).

The Prior Art Concerning Hedgehog/Smoothened Signaling and Molecules that Provide its Selective Inhibition

Described first in a publication of the results of a systematic screen of the genes that affect pattern formation during embryo development (Nüsslein-Volhard C et al, Nature 1980; 287:795-801), the hedgehog gene and the molecular signaling initiated by its product have been found to be largely conserved in species from drosophila to human. Hedgehog gene encodes for a secreted processed polypeptide (abbreviated here as Hh). Binding of Hh to a transmembrane protein, Patched, on a receiving cell initiates a molecular signaling transduced in the cell by another transmembrane protein, Smoothened (abbreviated here as Smo). When not liganded by Hh, Patched inhibits the signaling activity of Smo and the binding of Hh to Patched relieves the inhibition of Smo by Patched. The signaling by the relieved Smo has been determined to have a single end point in the cell, the Ci/Gli transcription factors that recognize a consensus sequence in the Hh target genes and affect their transcription (Method N et al, Development 2001; 128:733-742). The Smoothened protein has been determined to be essential for the signaling initiated by Hh in diverse species (Struhl G et al, Development 1997; 124:2155-2165; Wang Q T et al, Development 2000; 127:3131-3139; Zhang X M et al, Cell 2001; 105:781-792).

Besides the genetic means targeting Hh or Smo, several compounds have been purpose made for selective inhibition of Hh/Smo signaling in animals. Affinity-purified and monoclonal function-blocking anti-Hh antibodies have been made and shown to provide selective inhibition of Hh/Smo signaling in the administered embryos by multiple criteria (e.g. Ericson J et al, Cell 1996; 87:661-673). The brain in the vertebrate embryos that has loss of Hh expression shows inhibition of differentiation of various neural cells that are normally induced by Hh and the animals show consequent brain malformations that include a fusion of the developing eyes, called cyclopia (Krauss S et al, Cell 1993; 75:1431-1444).

Causation of such brain malformations and cyclopia and deaths of fetuses and mothers in the animals administered with the teratogenic Veratrum alkaloids cyclopamine or jervine had been determined in various vertebrate species (Keeler R F, Proceedings Of The Society For Experimental Biology and Medicine 1975; 149:302-306; Omnell M L et al, Teratology 1990; 42:105-119). Incardona I et al (Development 1998; 125:3553-3562) and Cooper M K et al (Science 1998; 280:1603-1607), using methods like in the earlier investigations of Ericson et al (ibid), described that exposure of developing chicken embryos to cyclopamine or jervine caused these brain malformations and cyclopia due to a direct and selective inhibition of Hh/Smo signaling in the animals. Administration of cyclopamine or jervine to the developing embryos was found to cause a phenocopy of a Hh loss-of-function mutation and several further test results showing a direct and selective inhibition of Hh/Smo signaling in the animals by these compounds were also described (Incardona et al, ibid; Cooper et al, ibid).

Automatable in vitro assays with a Gli recognition sequence-driven reporter have also been described and provide quantitative data about the inhibition of Hh/Smo signaling by candidate compounds rapidly (e.g. Sasaki H et al, Development 1997; 124:1313-1322). Using patched −/− cells in such an assay, Taipale J et al (Nature 2000; 406:1005-1009) described that cyclopamine inhibits Hh/Smo signaling downstream of Patched, at the level of Smo, and described a derivative of it that was found to be more potent in the same assay. Molecules of interest determined to inhibit Hh/Smo signaling in such in vitro screens can then be tested in an available animal model for suitability for selective inhibition of Hh/Smo signaling in animals. Gaffield W et al (Cellular and Molecular Biology 1999; 45:579-588) described results of such animal testing and selective inhibition of Hh/Smo signaling in the administered chicken embryos by cyclopamine and enhancement of the inhibitory activity by conversion of cyclopamine to its 4-ene-3-one derivative.

Methods employing developing chicken and other embryos as convenient tools have been widely used for determining whether or not a molecule of interest can be used for selective inhibition of Hh/Smo signaling in animals. Stenkamp D L et al (Developmental Biology 2000; 220:238-252) and Nasevicius A et al (Nature Genetics 2000; 26:216-220) have described that developing zebrafish provide a particularly suitable model due to the ease of observation of the effects of administered molecules and known Hh loss-of-function mutants. They have described purpose made new molecules for selective inhibition of Hh/Smo signaling and causation of such inhibition in the administered animals by multiple criteria, including the phenocopying of a loss-of-function mutation of Hh and selective inhibition of differentiation of various cell types in vivo that are normally induced by Hh (Stenkamp et al, ibid; Nasevicius et al, ibid). Treier M et al (Development 2001; 128:377-386) described use of a macromolecule (HIP) that selectively bound to Hh for selective inhibition of Hh/Smo signaling in vivo.

Hh and other proteins that take part in Hh/Smo signaling have been found to be expressed in adults of various species that have been investigated, including in human, throughout different tissues and organs (Hahn H et al, Journal of Biological Chemistry 1996; 271:12125-12128; Takabatake T et al, FEBS Letters 1997; 401:485-499; Traiffort E et al, European Journal of Neuroscience 1999; 11:3199-3214; Koebernick K et al, Mechanisms of Development 2001; 100:303-308).

Hh/Smo signaling has been described to be required for numerous normal functions in adults. Hair follicle epithelial cells that show continuity with the epidermal basal layer cells were found to show Hh/Smo signaling in adults and the hair cycle was found to be affected by Hh/Smo signaling (Sato N et al, Journal of Clinical Investigation 1999; 104:855-864). Hh/Smo signaling has been described to be required for normal stem cell functions in adults of various species (Zhang Y et al, Nature 2001; 410:599-604; Van der Eerden B C et al, Journal of Bone and Mineral Research 2000; 15:1045-1055; Detmer K et al, Blood Cells Molecules and Diseases 2000; 26:360-372). Detmer et al, ibid, described that formation of differentiated blood cells from CD34+ stem cells of adult human bone marrow is stimulated by Hh and that treatment of the cells in culture with cyclopamine blocked this effect.

The Prior Art Concerning the Skin Disorders that Show Inhibition of Differentiation of Epidermal Cells and Accompanying Features Like in Psoriasis

Inhibition of differentiation of epidermal cells in lesional skin and the consequently reduced epidermal barrier function have been known to be shared features of psoriasis and a group of related skin disorders that show in addition linkages to the same genetic loci that predispose to psoriasis and to that group of skin disorders. Elder J T et al (Archives of Dermatology 2001; 137:1447-1454) reviewing the genetic predisposition to psoriasis mention several loci that have been identified to predispose both to psoriasis and to atopic dermatitis. Cookson W O et al (Nature Genetics 2001; 27:372-373), whose findings are referred in the review by Elder et al, describe identification of loci at 1q21, 17q25 and 20p that show linkage to both psoriasis and atopic dermatitis and point out that the locus at 1q21 has already been shown to contain a cluster of genes that affect epidermal differentiation and that inhibition of differentiation is a characteristic of both of these conditions.

Bernerd F et al (Journal of Investigative Dermatology 1992; 98:902-910) described that inhibition of differentiation of epidermal cells is detectable in the epidermal basal layer of lesional skin by several criteria in the investigated psoriasis patients. The initiation of keratin 10 (K10) expression in a subpopulation of basal layer cells in normal skin was absent in lesional skin and the decrease or lack of K10 expression persisted also in the suprabasal layers that showed further molecular and morphological signs of inhibition of differentiation as well (Bernerd et al, ibid). A classical morphological sign of inhibition of epidermal differentiation, decrease or loss of formation of the granular layer cells, has been found to be accompanied by decreases of the expressions of the molecular markers of differentiation, such as K10 and filaggrin, in the lesional skin of patients having psoriasis and ichthyosis vulgaris, which is also a scaling skin disorder like psoriasis and these patients commonly show atopy (Nirunsiksuri W et al, Journal of Investigative Dermatology 1998; 110:854-861). Occurrence of the normal epidermal barrier function has been known to be dependent on the epidermal differentiation process that supplies the keratohyalin granules comprised of the aggregates of keratin filaments, filaggrin and other constituents of the permeability barrier that forms above the granular cells and the barrier function is known to be similarly impaired in lesional skin in patients having psoriasis, atopic dermatitis and ichthyosis vulgaris (Jensen J M et al, Journal of Investigative Dermatology 2000; 115:708-713).

### Principles of Drug Therapy Founded in the Prior Art

Extensive experience with patients given various drug treatments has shown that whereas drug treatments of symptoms may help patients in the absence of a solution otherwise, determination of the critical upstream events of pathogenesis that lead to the occurrence of a disease is often a precondition of development of a new drug treatment that is effective and safe for the patients and such a treatment can put end to multiple symptoms simultaneously. A further principle of drug treatment that has also been established in the art is that once a pathological process upstream and critical in the occurrence of a disease is determined, a pharmaceutically active compound that selectively intervenes with it without harming the patient through an effect or effects on the innumerable physiological processes in the patient must be used for a new drug treatment based on that determination.

A well-known example illustrative about these principles has been the drug treatments of peptic ulcer patients practiced prior to the determination that an infection by Helicobacter pylori is a critical upstream event in the pathogenesis of that disease. The previous drug treatments that attempted to decrease the gastric acidity to help to heal the ulcers and to alleviate gastric pain were poorly effective and were made mostly unneeded with the introduction of drug treatments that got rid of the H. pylori infection and ulcers. Whereas the nature of the target in that disease (a pathological process caused by a bacterium that is easy to selectively target in human body) has facilitated development of a safe and effective drug treatment of peptic ulcer disease, the basic principle of selectively intervening with an identified pathological process has been repeatedly confirmed as a precondition of being able to avoid the side effects due to the drug effects on unintended events in patients as reviewed and described in the scientific journal articles about the drug treatments of various diseases excerpted below.

Delyani J A (Kidney International 2000; 57:1408-1411) reviewed treatment of the aldosterone mediated cardiovascular disease as follows. “ . . . aldosterone . . . can mediate edema”. “ . . . elevated levels . . . result in interstitial cardiac fibrosis”. “The limited utility of spironolactone owing to the . . . side effects has been especially frustrating given the . . . role of aldosterone in cardiovascular disease. To obviate these limitations, eplerenone is . . . developed . . . Eplerenone is a competitive antagonist . . . with . . . excellent selectivity for the mineralocorticoid receptor”. Its “affinity is approximately 10- to 20-fold less than spironolactone for the aldosterone receptor. . . . However, unlike spironolacone, eplerenone has little affinity for other steroid receptors . . . there are no steroid-related adverse effects . . . phase I trials indicated . . . a good safety profile . . . effective in hypertension as well as heart failure”.

Weldon M J et al (Gut 1994; 35:867-871) reviewed treatment of inflammatory bowel disease as follows. “Greater understanding of inflammatory bowel disease, and . . . of the central role of activated T cells, has prompted a search for drugs”. “The goal is to provide more effective and less toxic therapy by developing treatment targeted to specific . . . effector mechanisms”. “More selective targeting of activated T cells is therefore needed. Since activated T cells in inflammatory bowel disease . . . express αIL-2r whereas . . . resting T cells do not, antibodies to this receptor would provide such selectivity”.

Ellis C N et al (New England Journal of Medicine 2001; 345:248-255) described a new drug treatment of psoriasis on the basis of the knowledge in prior art about the occurrence of psoriasis lesions as follows. “Psoriatic plaques are characterized by infiltration with CD45RO+ memory effector T lymphocytes”. “ . . . LFA-3-CD2 signal plays an important part in the activation of T lymphocytes”. “ . . . CD45RO+ T lymphocyte subgroups . . . contain the clonal precursors driving the pathogenic process”. “Alefacept selectively targets CD45RO+ memory effector T lymphocytes”. “ . . . alefacept . . . was designed to prevent the interaction between LFA-3 and CD2”. “ . . . patients receiving alefacept had a greater decrease in the psoriasis area-and-severity index than those receiving placebo”.

Timermans PBWM (Hypertension Research 1999; 22:147-153) reviewed treatment of angiotensin II receptor type 1 mediated hypertensive disease as follows. “Activation of RAAS is critically involved in the development and maintenance of hypertension and congestive heart failure . . . Ang II . . . is the primary mediator of the RAAS”. “ . . . selective . . . Ang II type 1 (AT1) receptor antagonists provided . . . benefits . . . avoid the nonspecificity of the Ang I converting enzyme . . . inhibitors”. “ . . . all of the known actions of Ang II could be blocked by losartan, emphasizing the major role of the AT1 . . . in the patho(physiological) actions of this hormone . . . it also clearly explains why most of the pharmaceutical effort has been focused on developing . . . AT1 . . . selective antagonists”.

Culman J (Experimental Physiology 2000; 85:757-767) reviewed uses of purpose-made antisense oligonucleotide compounds in drug treatment as follows. “ . . . classical pharmacologic approaches . . . are often based on the inhibition of biologically active proteins”. “Binding of antisense oligonucleotides to the complementary . . . sequence . . . results in a selective inhibition of transcription or translation . . . This . . . represents a promising basis for . . . therapies”. “ . . . an important advantage of antisense strategy is . . . the ability to selectively inhibit the expression of biologically active proteins where . . . agents are not available or show limited selectivity”.

Pelaia G et al (Allergy 2000; 55 (Supplement 61):60-66) reviewed drug treatment of asthma as follows. “ . . . adenosine induces bronchoconstriction via stimulation of A1-receptors”. “Respirable antisense oligonucleotides . . . have been designed which hybridize to A1-receptor . . . thereby . . . selectively reducing A1-receptor number”. Reviewing the knowledge about the pathogenesis of asthma, they added “These new therapeutic approaches have the advantage . . . of being more specifically targeted on the pathogenetic events”. “ . . . all sharing a common basic principle; that is, to develop drugs more directly targeted on the pathophysiology of the disease”.

These descriptions of the drug treatments of patients having various diseases in scientific publications by independent scientists all emphasize the aforementioned same basic medical principles that have been established in the art and show also their rationale with examples.

### SUMMARY OF THE INVENTION

This invention concerns the use of cyclopamine, a naturally occurring steroidal alkaloid known for over thirty years, for the treatment of psoriasis and achievement of rapid clearance of the psoriatic skin lesions together with the reversion of the histopathological signs of disease to normalcy with no detectable side effects. The cyclopamine-induced clearance of psoriatic lesions from the skin of patients is associated with the causation of cellular differentiation in lesional epidermis and with the rapid disappearance of CD4(+) lymphocytes and other inflammatory cells from lesional skin. Therapeutic compositions comprising of cyclopamine and a corticosteroid and/or the pre-treatment of lesions with a corticosteroid provide significantly further increased therapeutic effectiveness over the use of cyclopamine alone or a corticosteroid alone (the latter displays insignificant or marginal effectiveness when used for less than a week). Cyclopamine, a known selective inhibitor of Hedgehog/Smoothened signaling (Incardona J P et al, Development 1998; 125:3553-3562; Cooper M K et al, Science 1998; 280:1603-1607), can be replaced by another selective inhibitor of Hedgehog/Smoothened signaling such as a functionally equivalent derivative of cyclopamine in a pharmaceutical composition for the described treatment.

According to one aspect, the present invention is directed to the use of cyclopamine or a pharmaceutically acceptable salt or a derivative thereof for clearing CD4 positive lymphocytes from psoriatic lesions in human patients.

In a second aspect, the invention is directed to the use of cyclopamine or a pharmaceutically acceptable salt or a derivative thereof for causation of the differentiation of the epidermal cells in psoriatic lesions in human patients.

In a further aspect, the invention is directed to the use of cyclopamine or a pharmaceutically acceptable salt or a derivative thereof for restoring the decreased or lost cytokeratin 15 expression in the epidermal basal layer of psoriatic lesional skin in human patients, and/or the use of cyclopamine or a pharmaceutically acceptable salt or a derivative thereof for attaining decrease or disappearance of erythema from psoriatic lesional skin in human patients within 12 hours of application.

Preferably, regression and clearance of psoriatic lesions are obtained within 1-8 days, without harming the non-lesional skin. Skin lesions in other skin disorders associated with impaired differentiation of epidermal cells like in psoriasis also show likewise regression and disappearance by the described treatment. With therapeutic compositions comprising of cyclopamine or a pharmaceutically acceptable salt or derivative thereof and a corticosteroid and/or pre-treatment of lesions with a cortiscosteroid, regression and clearance of psoriatic lesions are obtained within 1-4 days, commonly within 1-2 days. Most preferably, treatment for a day with therapeutic compositions comprising of cyclopamine or a pharmaceutically acceptable salt or a derivative thereof and a cortiscosteroid provides regression and clearance of psoriatic lesions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows appearance of a psoriatic lesion (about 11×13 mm) at the dorsum of hand of a 57-year old man prior to the application of treatment.

FIG. 1B shows the same lesion as in FIG. 1 A with the cyclopamine cream applied to its proximal half (upper in the figure) and covered against accidental smearing and loss.

FIG. 1C shows the same lesion as in FIG. 1 A at the 24th hour of exposure to the cyclopamine cream. Regression of the psoriatic plaque from the cyclopamine-treated proximal half (upper in the figure) is evident.

FIG. 1D shows appearance of a psoriatic lesion (about 7×9 mm) at the left scapular region of a 54 year old man prior to the application of cyclopamine.

FIG. 1E shows the same skin area as in FIG. 1A after 1 day of treatment and 7 days of follow-up. With the possible exception of slight erythema, lesion is no longer visible.

FIG. 1F shows the same skin area as in FIG. 1 E on the 14th day of follow-up. No lesion is visible, skin appears normal.

FIG. 2A to FIG. 2W show skin tissue sections from the non-lesional skin, the non-treated psoriatic lesional skin and the cyclopamine-treated lesional skin of the patient presented in FIG. 1A to FIG. 1C.

FIG. 2A shows a section from the non-lesional skin tissue. Hematoxylene-Eosine (H&E) staining, 200× original magnification.

FIG. 2B shows a tissue section from the non-treated psoriatic lesional skin. H&E, 100× original magnification.

FIG. 2C shows a tissue section from the cyclopamine-treated half of the psoriatic lesion at the 24th hour. H&E, 200× original magnification.

FIG. 2D shows another region from the cyclopamine-treated half of the psoriatic lesion at the 24th hour at 400× original magnification (H&E).

FIG. 2E shows a tissue section from the cyclopamine-treated and non-treated junctional area of the psoriatic lesion at the 24th hour. H&E, 200× original magnification.

FIG. 2F shows another tissue section from the cyclopamine-treated and non-treated junctional area of the psoriatic lesion at the 24th hour at 100× original magnification (H&E). The area of the psoriatic lesion covered under the applicator with the cyclopamine cream is towards the left of figure (left of the indentation).

FIG. 2G shows non-lesional skin tissue with immunohistochemically detected Ki-67 antigen. 400× original magnification. Ki-67 displaying cells in the epidermis are seen to be restricted to the basal layer.

FIG. 2H shows non-treated psoriatic lesional skin tissue with immunohistochemically detected Ki-67 antigen. 200× original magnification. Numerous Ki-67 displaying cells are seen in the suprabasal layers of epidermis.

FIG. 2I shows a tissue section from the cyclopamine-treated half of the psoriatic lesion at the 24th hour with immunohistochemical staining for the Ki-67 antigen. 200× original magnification. Return of the Ki-67 displaying cells to the epidermal basal layer is seen.

FIG. 2J shows a tissue section from the cyclopamine-treated and non-treated junctional area of the psoriatic lesion at the 24th hour with immunohistochemical staining for the Ki-67 antigen. 200× original magnification. The cyclopamine-receiving tissue is towards the left of figure.

FIG. 2K shows a Ki-67 stained tissue section from the junctional area of the non-lesional skin with the cyclopamine-treated half of the psoriatic lesion at the 24th hour. 100× original magnification. The non-lesional skin is towards the left of figure.

FIG. 2L shows non-lesional skin tissue stained immunohistochemically for the epithelial antigen by the Ber-EP4 antibody. 100× original magnification. Epidermal basal layer cells are seen to display the epithelial antigen.

FIG. 2M shows non-treated psoriatic lesional skin tissue stained immunohistochemically for the epithelial antigen using the Ber-EP4 antibody. 100× original magnification. The Ber-EP4 detectable epithelial antigen is seen to be greatly decreased to non-existent in the psoriatic lesional epidermis.

FIG. 2N shows a tissue section from the cyclopamine-treated half of the psoriatic lesion at the 24th hour, stained immunohistochemically for the epithelial antigen using the Ber-EP4 antibody. 400× original magnification. Epidermal basal layer cells are seen to display the epithelial antigen.

FIG. 2O shows a tissue section from the cyclopamine-treated and non-treated junctional area of the psoriatic lesion at the 24th hour, stained immunohistochemically for the epithelial antigen by the Ber-EP4 antibody. 100× original magnification. The area of the psoriatic lesion covered under the applicator with the cyclopamine cream is towards the left of figure (left of the indentation).

FIG. 2P shows a tissue section from the junctional area of the non-lesional skin with the cyclopamine-treated half of the psoriatic lesion at the 24th hour. Immunohistochemical staining for the epithelial antigen using the Ber-EP4 antibody is shown at 100× original magnification. Non-lesional skin is towards the left of figure.

FIG. 2R shows non-lesional skin tissue stained immunohistochemically with the C8/144B antibody that binds the human CD8 antigen and the cytokeratin 15. 400× original magnification.

FIG. 2S shows a tissue section from the junctional area of the non-lesional skin with the non-treated lesional skin. Immunohistochemical staining with the C8/144B antibody shown at 100× original magnification. Non-lesional skin is towards the left of figure.

FIG. 2T shows a tissue section from the cyclopamine-treated half of the psoriatic lesion at the 24 h hour, stained immunohistochemically using the C8/144B antibody. 400× original magnification.

FIG. 2U shows a tissue section from the junctional area of the non-lesional skin with the cyclopamine-treated lesional skin at the 24th hour, stained immunohistochemically using the C8/144B antibody. 100× original magnification. Non-lesional skin is towards the left of figure.

FIG. 2V shows a tissue section from the non-treated psoriatic lesional skin stained immunohistochemically with the MT310 antibody that binds the human CD4 antigen. 100× original magnification. Abundant CD4 positive lymphocytes are seen to infiltrate the dermis.

FIG. 2W shows a tissue section from the junctional area of the cyclopamine-treated and non-treated lesional skin at the 24th hour, stained immunohistochemically with the MT310 antibody. 40× original magnification. Disappearance of the CD4 positive lymphocytes from the dermis of treated area (towards the left of figure) are seen.

FIG. 3A to FIG. 3L show skin tissue sections from the non-lesional skin, the non-treated lesional skin and the cyclopamine-treated lesional skin of various patients (age range 29 years to 57 years and the range of disease duration 1.5 years to 6 years) with psoriasis vulgaris.

FIG. 3A shows non-lesional skin tissue of a patient with psoriasis. H&E, 200× original magnification.

FIG. 3B shows a tissue section from the non-treated psoriatic lesional skin of the same patient as in FIG. 3A. H&E, 200× original magnification.

FIG. 3C shows a tissue section from the cyclopamine-treated psoriatic lesion of the same patient as in FIG. 3A at the 24th hour of treatment. H&E, 200× original magnification.

FIG. 3D shows immunohistochemical staining for the Ki-67 antigen of a non-lesional skin tissue section from a patient with psoriasis vulgaris. 400× original magnification.

FIG. 3E shows immunohistochemical staining for the Ki-67 antigen of a tissue section from a non-treated psoriatic lesion of the same patient as in FIG. 3D. 400× original magnification. Numerous Ki-67 displaying cell are seen in the suprabasal layers in epidermis.

FIG. 3F shows immunohistochemical staining for the Ki-67 antigen of a tissue section from a cyclopamine-treated psoriatic lesion of the same patient as in FIG. 3D at the 24th hour of treatment. 400× original magnification. Return of the Ki-67 displaying cells to the epidermal basal layer is seen.

FIG. 3G shows non-lesional skin tissue of a patient with psoriasis stained immunohistochemically for the epithelial antigen using the Ber-EP4 antibody. 100× original magnification.

FIG. 3H shows a tissue section from a non-treated psoriatic lesional skin of the same patient as in FIG. 3G stained immunohistochemically for the epithelial antigen using the Ber-EP4 antibody. 100× original magnification.

FIG. 3I shows a tissue section from a cyclopamine-treated psoriatic lesion of the same patient as in FIG. 3G at the 24th hour of treatment, stained immunohistochemically for the epithelial antigen using the Ber-EP4 antibody. 100× original magnification.

FIG. 3J shows non-lesional skin tissue of a patient with psoriasis stained immunohistochemically with the C8/144B antibody that binds the human CD8 antigen and the cytokeratin 15. 100× original magnification.

FIG. 3K shows a tissue section from a non-treated psoriatic plaque of the same patient as in FIG. 3J stained immunohistochemically with the C8/144B antibody that binds the human CD8 antigen and the cytokeratin 15. 200× original magnification.

FIG. 3L shows a tissue section from a cyclopamine-treated psoriatic lesion of the same patient as in FIG. 3J at the 24th hour of treatment stained immunohistochemically with the C8/144B antibody that binds the human CD8 antigen and the cytokeratin 15. 200× original magnification.

FIG. 4A shows appearance of a psoriatic lesion (approximately 7×8 mm) in the antecubital region of a 29-year old man prior to treatment.

FIG. 4B shows the same lesion as in FIG. 4B after treatment with the cyclopamine cream for 24 hours and follow-up without treatment for 44 hours (68th hour from the onset of treatment).

FIG. 4C shows a psoriatic lesion (approximately 9×11 mm) in the deltoid region of a 29-year old man prior to treatment.

FIG. 4D shows the same lesion as in FIG. 4C on the 68th hour of treatment with a pre-mixed cream containing equal volumes of the cyclopamine cream and a cream preparation of clobetasol 17-propionate (0.5 mg/g).

FIG. 4E shows a psoriatic lesion (approximately 11×12 mm) in the hypochondrial region of a 29-year old man after 48 hours of treatment with a cream preparation of clobetasol 17-propionate (0.5 mg/g).

FIG. 4F shows the same lesion as in FIG. 4E on the 24th hour of switching the treatment from clobetasol 17-propionate (0.5 mg/g) to the cyclopamine cream.

FIG. 4G shows a tissue section from the junctional area of non-treated psoriatic lesional skin (to the left of figure) and the adjacent non-lesional skin (to the right of figure) with immunohistochemical staining for the CD44 antigen. Expression of CD44, which increases normally with the differentiation of epidermal keratinocytes, is seen to be significantly decreased in the suprabasal and spinous cells of psoriatic lesional skin in comparison to the situation in non-lesional epidermis. 50× original magnification.

FIG. 4H shows higher magnification (200×) view of a section from the non-treated psoriatic lesional skin with immunohistochemical staining for the CD44 antigen. Expression of CD44 in the suprabasal and spinous layers is seen to be decreased in comparison to the situation in non-lesional epidermis.

FIG. 4I shows a tissue section from the lesional skin with immunohistochemical staining for the CD44 antigen on the 24th hours of treatment with cyclopamine. 200× original magnification. Notice the increase and normalization of the expression of CD44 following treatment with cyclopamine (compare to the non-treated lesional skin in FIG. 4H) as well as the near normalization of epidermal hyperplasia.

FIG. 4J shows non-lesional skin tissue with immunohistochemically detected EGFR. The EGFR displaying cells in the epidermis are seen to be restricted to the basal layer. 200× original magnification.

FIG. 4K shows non-treated psoriatic lesional skin tissue with immunohistochemically detected EGFR. Suprabasal cells in the lesional epidermis are seen to show markedly increased EGFR expression. 200× original magnification.

FIG. 4L shows psoriatic lesional skin tissue that was treated with the cyclopamine cream for 24 hours before excision and then stained immunohistochemically for EGFR. The EGFR displaying cells in the epidermis are seen to be restricted to the basal layer. 200× original magnification.

FIG. 5A shows palm of a palmoplantar pustulosis patient prior to treatment.

FIG. 5B shows foot of the same palmoplantar pustulosis patient as in FIG. 5A and shows sparing of the non-plantar skin.

FIG. 5C shows palm of the patient shown in FIG. 5A at the end of day 4 of treatment with a cream comprised of cyclopamine.

FIG. 5D shows the palm shown in FIG. 5C after a non-treated period of about a month following the 4-day treatment.

FIG. 5E shows the palm shown in FIG. 5D after a non-treated period of about 10 days following a treatment for 40 hours.

FIG. 6A shows an involved nail of a palmoplantar pustulosis patient prior to treatment. This is the same patient whose palm is shown in FIG. 5A.

FIG. 6B shows the thumb and nail shown in FIG. 6A on day 4 of treatment with a cream comprised of cyclopamine.

FIG. 6C shows the thumb and nail shown in FIG. 6B after a non-treated period of about five weeks following the 4-day treatment. The treatment is seen to have provided healthy normal nail growth during this period (proximal of the nail).

### COLOR PRINTS

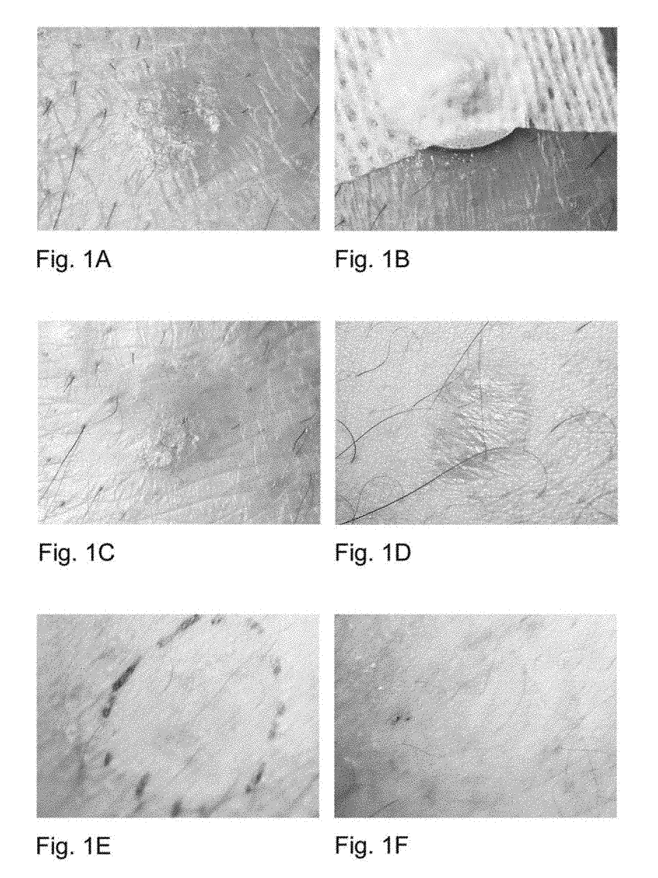

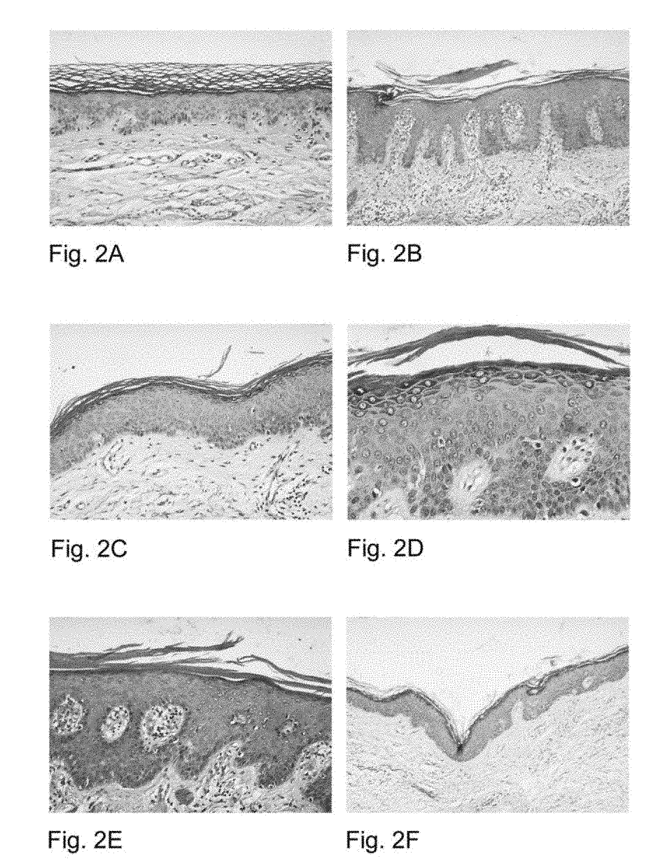

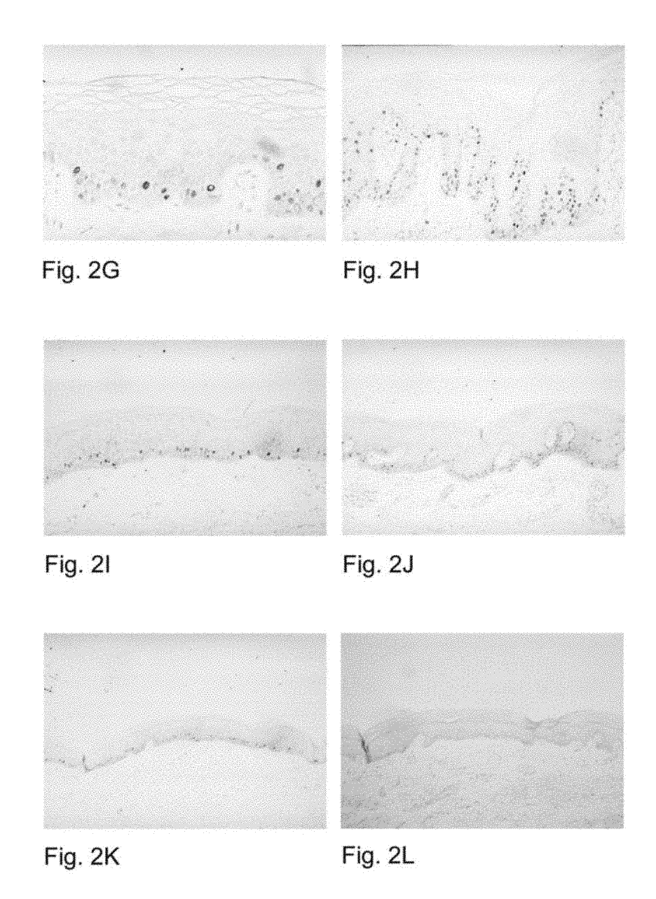

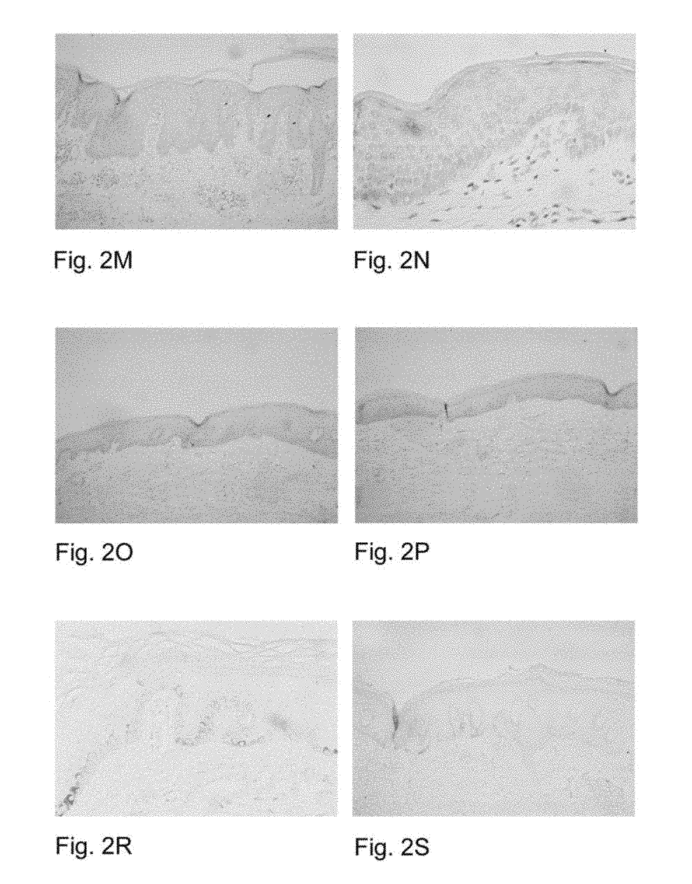

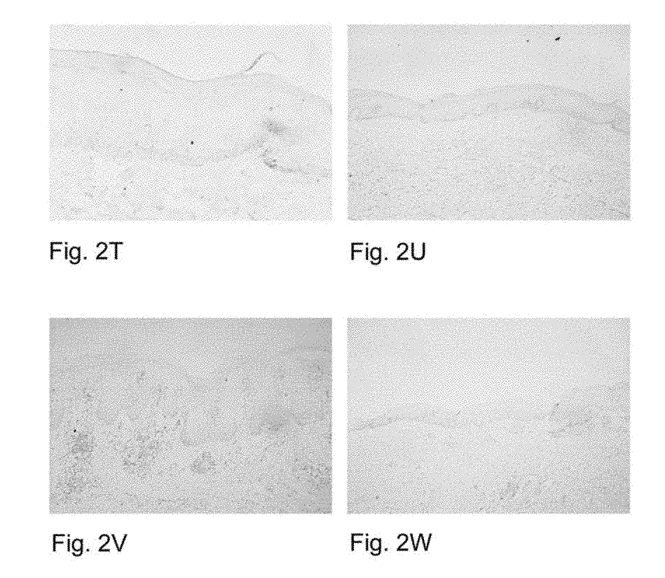

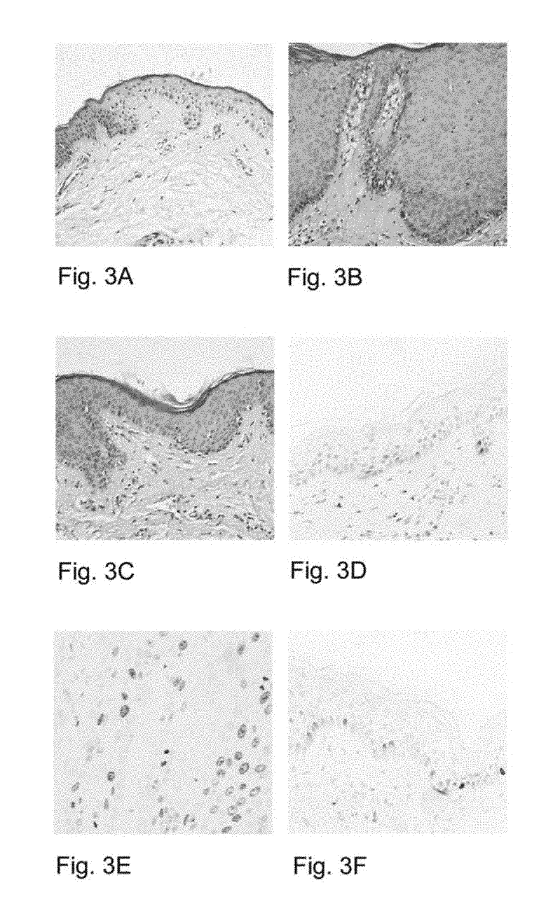

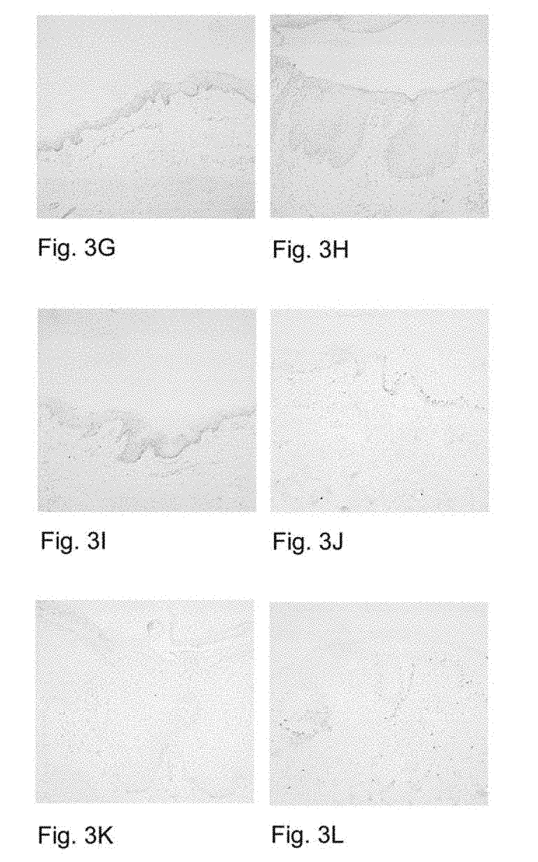

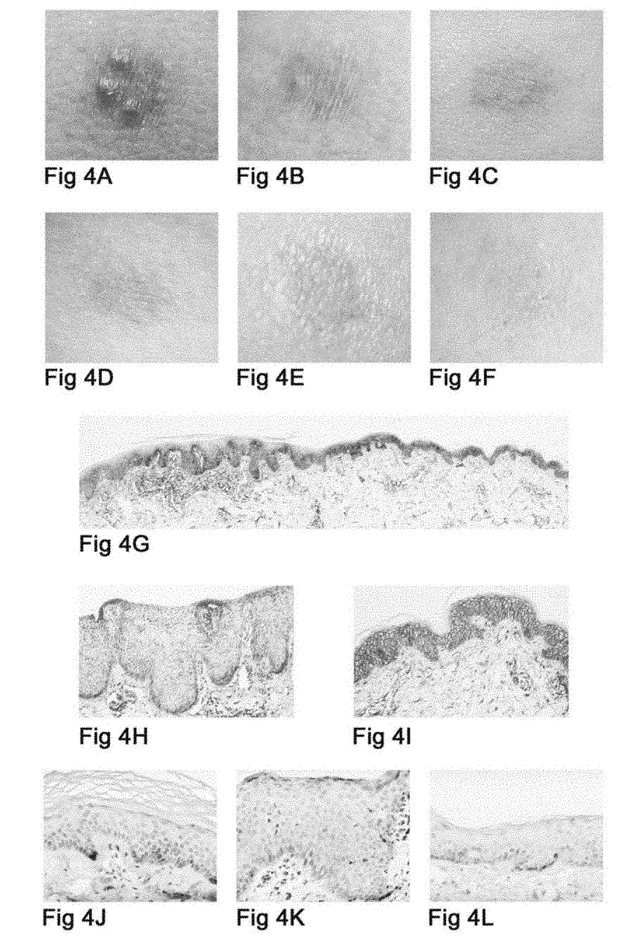

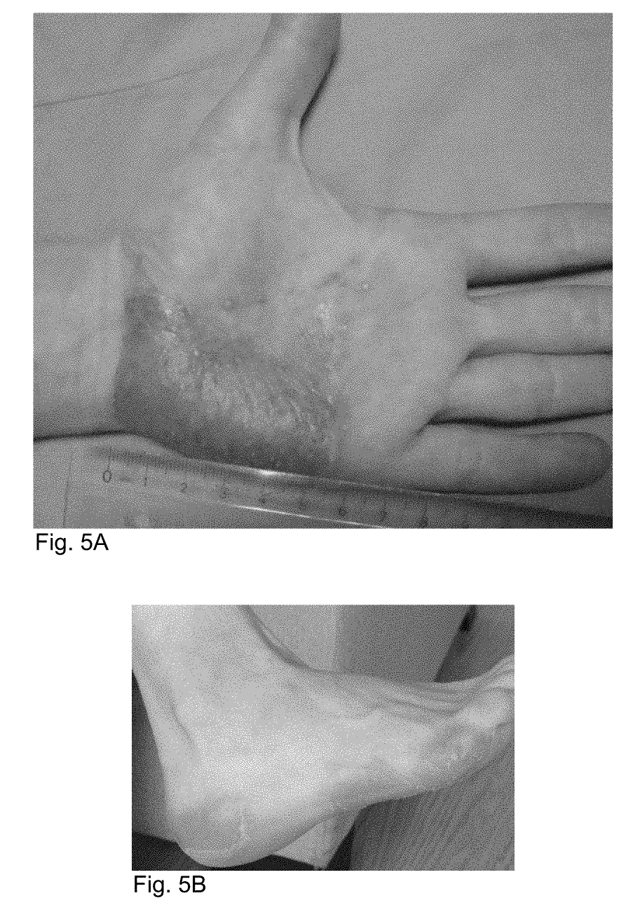

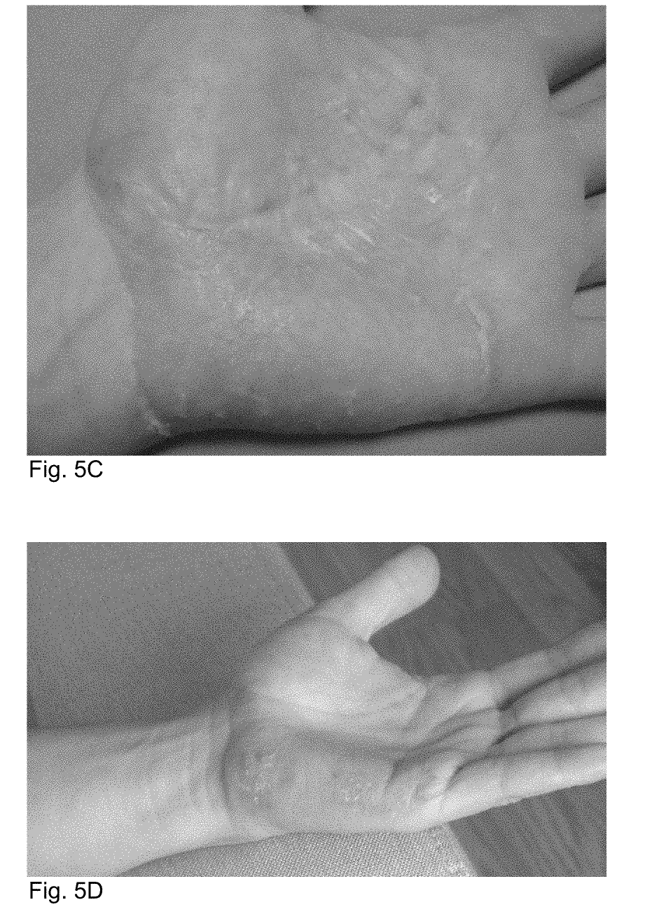

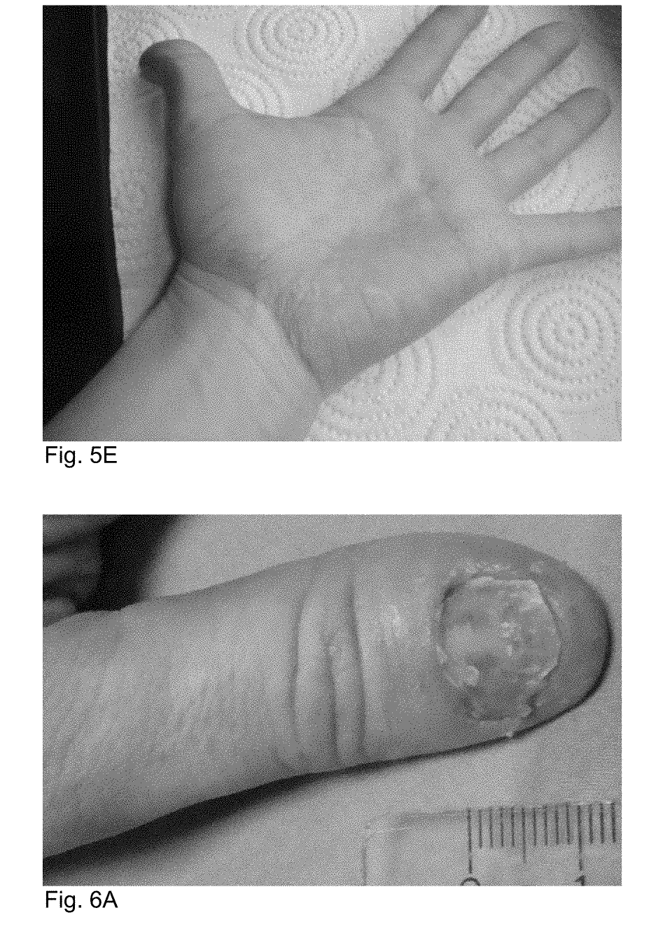

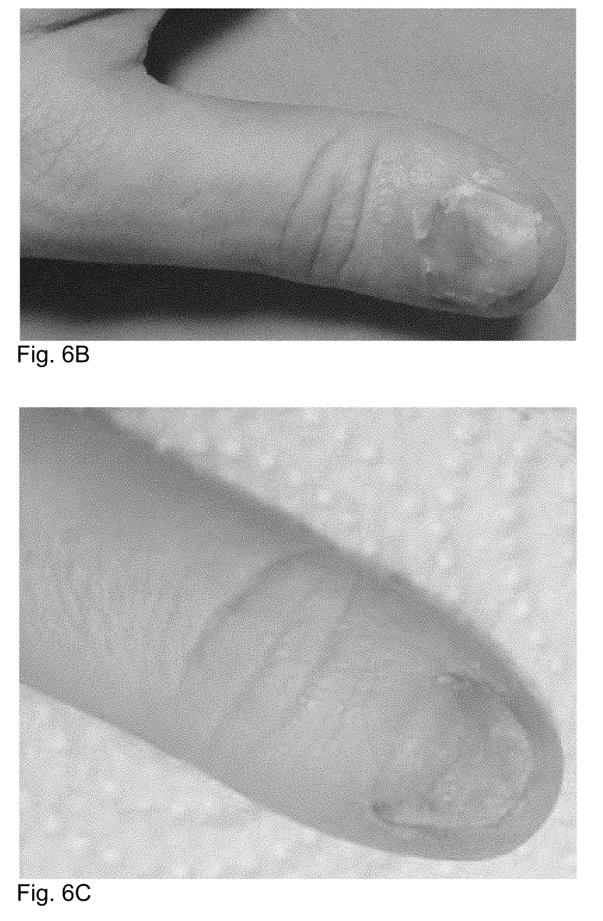

Color prints of the same figures as on pages 1/12, 2/12, 3/12, 4/12, 5/12, 6/12, 7/12, 8/12, 9/12, 10/12, 11/12, 12/12 (FIG. 1A, FIG. 1B, FIG. 1C, FIG. 1D, FIG. 1D, FIG. 1E, FIG. 1F, FIG. 2A, FIG. 2B, FIG. 2C, FIG. 2D, FIG. 2E, FIG. 2F, FIG. 2G, FIG. 2H, FIG. 2I, FIG. 2J, FIG. 2K, FIG. 2L, FIG. 2M, FIG. 2N, FIG. 2O, FIG. 2P, FIG. 2R, FIG. 2S, FIG. 2T, FIG. 2U, FIG. 2V, FIG. 2W, FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L, FIG. 5A, FIG. 5B, FIG. 5C, FIG. 5D, FIG. 5E, FIG. 6A, FIG. 6B, FIG. 6C), added as pages 1/12a, 2/12a, 3/12a, 4/12a, 5/12a, 6/12a, 7/12a, 8/12a, 9/12a, 10/12a, 11/12a, 12/12a because the immunohistochemical data and findings, due to their nature, can be conveyed best in color rather than in gray-scale; we respectfully request consideration of this fact by the Patent Authority and the keeping of the pages as part of this patent application. However, the pages 1/12a, 2/12a, 3/12a, 4/12a, 5/12a, 6/12a, 7/12a, 8/12a, 9/12a, 10/12a, 11/12a, 12/12a may be removed from the patent application if it is deemed necessary by the Patent Authority.

### DESCRIPTION OF THE INVENTION

This invention relates to the use of cyclopamine, a naturally occurring steroidal alkaloid known for over thirty years, for the treatment of psoriasis and the achievement of rapid clearance of psoriatic lesions from the patient skin as fast as within a day with no detectable side effects. Disappearances of the clinical signs of psoriasis, including the erythema and scaling, from the skin of patients are accompanied by the reversions of the histopathological signs of psoriasis to normalcy and are achievable by topical treatment. Follow-up of the treated skin areas shows healthy-looking normal skin over months. These features make the use of cyclopamine highly desirable in the treatment of psoriasis and provide a solution to the long-standing problem of psoriasis treatment. Skin lesions in patients having a skin disorder other than psoriasis that also shows impaired differentiation of the epidermal cells in lesional skin are also found to show likewise regression and disappearance by the described treatment. Cyclopamine is known to be a selective inhibitor of Hedgehog/Smoothened signaling and it can be replaced by another selective inhibitor of the signaling such as a functionally equivalent derivative of cyclopamine in a pharmaceutical composition for the treatment. Therapeutic compositions comprising of cyclopamine or a pharmaceutically acceptable salt or a derivative thereof and a corticosteroid and/or the pre-treatment of lesions with a corticosteroid provide significantly further increased therapeutic effectiveness over the use of either alone.

For topical applications, cyclopamine can be dissolved in ethanol or another suitable solvent and mixed with a suitable base cream, ointment or gel or a foam preparation. Cyclopamine may also be entrapped in hydrogels or in other pharmaceutical forms enabling controlled release and may be adsorbed onto dermal patches. In a pharmaceutical preparation for topical administration, the cyclopamine or a pharmaceutically acceptable salt or derivative thereof should be present in a concentration of 0.001 mM to 100 mM, preferably in a concentration of 9 mM to 24 mM. The effects shown in figures FIG. 1A to FIG. 1F (clinical pictures) and FIG. 2A to FIG. 2W and FIG. 3A to FIG. 3L (histopathological and immunohistochemical findings) have been obtained by a cream preparation obtained by mixing a solution of cyclopamine in ethanol with a base cream so as to get a final concentration of 18 mM cyclopamine in cream. The base cream used is made predominantly of heavy paraffin oil (10% w/w), vaseline (10% w/w), stearyl alcohol (8% w/w), polyoxylsteareth-40 (3% w/w) and water (68% w/w) but another suitably formulated base cream is also possible. Optimal concentration of cyclopamine in a pharmaceutical form as well as the optimal dosing and application schedules can obviously be affected by such factors as the particular pharmaceutical form and the localization and characteristics of the skin lesion; however these can be determined by following well known published optimization methods. The dosing and the application schedules followed for the lesion shown in FIG. 1A (psoriatic plaque on the dorsum of hand) are as follows: about 30 μl cream applied directly onto the lesion with the aid of a steel spatula every four hours for 24 hours. The cream-applied area is protected from accidental smearing and loss of the cream by covering with an aluminum applicator (FIG. 1 B). The dosing and the application schedules followed for the lesion shown in FIG. 1 D (psoriatic plaque at the scapular region) are as follows: about 30 μl cream applied directly onto the lesion every four hours for 24 hours. The cream-applied area is similarly covered against the smearing and loss of cream. Other treated psoriatic patients and lesions received from about 30 to 35 μl cream to each lesion at intervals of every 3 to 5 hours, as suitable and convenient. Lesions were similarly covered against the smearing and loss of cream. Placebo cream (the base cream mixed with ethanol with no added cyclopamine) applications onto comparably sized psoriatic plaques as the cyclopamine-treated psoriatic plaques followed the same dosing, schedule and covering of the lesions; placebo-treated psoriatic plaques showed no detectable effect or regression (data not shown). Preservation of the undifferentiated cells in the normal epidermis and in hair follicles following exposure to cyclopamine, as described in an earlier patent application by us (Taş S. et al. (2001) PCT/TR 01/00027) as well as in this invention, provide information about the tolerable doses in other possible modes of administration of cyclopamine; e.g. intralesional injections or covering with suitable dermal patches and timed-release formulations or systemic administration of aqueous solutions or of cyclopamine entrapped in liposomes. In preparing therapeutic compositions comprising of cyclopamine and a corticosteroid, mixing of the two in the same pharmaceutical carrier is facilitated by their similar molecular structures (both being steroidal molecules) and by their similar lipid versus water solubility characteristics. Optimal concentrations of cyclopamine and a corticosteroid in a pharmaceutical form can obviously be affected by such factors as the particular pharmaceutical form and the particular corticosteroid and the mode of administration; however, these can be determined by following well known published methods of optimization. The therapeutic effect shown in FIG. 4D was obtained by a cream preparation comprising of about 9 mM cyclopamine and about 0.55 mM clobetasol 17-propionate in base cream made predominantly of heavy paraffin oil (10% w/w), vaseline (10% w/w), stearyl alcohol (8% w/w), polyoxysteareth-40 (3% w/w) and water (68% w/w). The corticosteroid used for treatment of the lesion shown in FIG. 4E was clobetasol 17-propionate in a final concentration of about 1.1 mM in base cream. Similar corticosteroid actions are obtained by substituting the clobetasol 17-propionate in this cream with about 50 mM hydrocortisone. In a pharmaceutical preparation comprising of cyclopamine or a pharmaceutically acceptable salt or derivative thereof and a corticosteroid, cyclopamine or a pharmaceutically acceptable salt or derivative thereof should exist in a concentration of 0.001 mM to 100 mM, preferably in a concentration of 9 mM to 24 mM, and clobetasol 17-propionate should be present in a concentration of 0.2 mM to 1.5 mM. Other corticosteroid molecules well known in the art are contemplated to be also suitable and capable of replacing the above-mentioned corticosteroid molecules at suitable concentrations (the suitable concentration ranges for corticosteroids are also known in the art).

The unprecedented therapeutic effectiveness of the treatment described herein is based on highly reproducible biological effects associated with the specific molecular and cellular changes that are also described herein. It is therefore specifically contemplated that other molecules can be derived from cyclopamine or synthesized in such a way that they exert similar receptor-binding properties and biological and therapeutic effects as cyclopamine. Such molecules are called here as “derivatives of cyclopamine”. The term “derivatives of cyclopamine”, as used here, is defined as follows:

A molecule that contains the region of cyclopamine molecule involved in the binding of cyclopamine to its biological target but contains in addition modifications of the parent cyclopamine molecule in such ways that the newly derived molecule continues to be able to bind specifically to the same biological target (i.e. the smoothened protein) to exert the biological effects of cyclopamine disclosed in this invention. Such modifications of cyclopamine may include one or more permissible replacement of or deletion of a molecular group in the cyclopamine molecule or addition of a molecular group (particularly a small molecular group such as the methyl group) to the cyclopamine molecule provided that the resultant molecule is stable and possesses the capability of specific binding to the same biological target as cyclopamine to exert the biological effects of cyclopamine disclosed in this invention. Derivation of such new molecules from cyclopamine can be readily achieved by those skilled in the art and the continuance or abolishment of the possession of the biological effects of cyclopamine in the newly derived molecule can also be readily determined by those skilled in the art, for example by testing for the biological effects disclosed in this application.

FIG. 1A and FIG. 1C show a psoriatic plaque before and after exposure to cyclopamine and the rapid clinical regression. The cyclopamine cream was applied to the proximal half of this lesion present on the dorsum of right hand of a 57 years old patient having plaque-type psoriasis. At intervals of 4 hours, approximately 30 μl cream was applied directly onto the lesion and the cream-applied region was covered against accidental smearing and loss of the cream (FIG. 1 B). Already on the 4th hour of treatment, the cream-applied half of the psoriatic plaque displayed slight decrease in erythema. The erythema was no longer visible in the cyclopamine-applied half of the lesion at the 12th hour and on the 24th hour, when the erythema and scaling had visibly disappeared from the cyclopamine-treated half (FIG. 1 C), the skin area corresponding to the entire former lesion (both the treated and non-treated halves) was excised together with an approximately 5 mm margin of surrounding non-involved skin.

FIG. 2A to FIG. 2W show histopathological and immunohistochemical examination findings of the tissue sections from the non-lesional skin, cyclopamine-treated lesional skin and non-treated lesional skin. Compared to the non-lesional skin (FIG. 2A), the non-treated lesional skin (FIG. 2B) is seen to display the typical histopathological signs of a psoriatic skin lesion mentioned above in the “Background of Invention”. Tissue sections from the cyclopamine-treated half of the original lesion reveal, however, dramatic improvement and regression to normalcy. These histopathological signs of regression to normalcy, exemplified in the figures FIG. 2C and FIG. 2D (examples from other cyclopamine-treated lesions and patients are given later) include the following:

Return of the thickened and elongated rete ridges to normal levels and marked decrease of epidermal hyperplasia (FIG. 2C versus FIG. 2B).

Return of the thinning of the suprapapillary epidermis to normalcy and the disappearance of papillary edema (FIG. 2C versus FIG. 2B).

Vigorous re-appearance of the granular layer of epidermis in the cyclopamine-treated epidermis (FIG. 2C and FIG. 2D) contrasted with the focally decreased or lost granular layer in the non-treated lesional epidermis (FIG. 2B).

Disappearance from the cyclopamine-treated lesional skin of most of the inflammatory cells that infiltrated the subepidermal dermis of the non-treated lesional skin (FIG. 2C versus FIG. 2B).

The hyperkeratosis and parakeratosis seen in the stratum corneum of the non-treated lesional skin (FIG. 2B) decreased but full normalization of this oldest epidermal layer did not yet happen at the 24th hour of cyclopamine treatment (FIG. 2C, FIG. 2D).

Tissue sections from the junctional area of the cyclopamine-treated and non-treated lesional skin revealed that regions of the lesional skin that received relatively lesser concentrations of cyclopamine (by diffusion from the nearby treated area) still displayed signs of regression towards normalcy but relatively less pronouncedly (FIG. 2E, FIG. 2F).

Relevant immunohistochemical findings with the tissues described above are summarized below and exemplified through figures FIG. 2G to FIG. 2W. All antibodies and reagents for these immunohistochemical investigations were purchased from DAKO (Glostrup, Denmark); human Ki-67 antigen was detected by the monoclonal antibody M7187, human epithelial antigen was detected by the monoclonal antibody Ber-EP4, human CD4 antigen was detected by the monoclonal antibody M0716, human CD8 antigen was detected by the monoclonal antibody C8/144B. Besides the CD8 antigen, the monoclonal antibody C8/144B is known to recognize and bind to the cytokeratin 15 expressed by the hair stem cells (Kanitakis J. et al. (1999) Eur. J. Dermatol. 9:363-365). A DAKO “universal visualization kit” (LSAB2) employing biotinylated secondary antibody and peroxidase-conjugated streptavidin (pre-diluted to match the dilutions of DAKO-supplied primary antibodies) was used for the visualization reactions. All reaction conditions were as recommended by the manufacturer.

The Ki-67 antigen is a marker of the proliferating cells. As shown in FIG. 2G, the Ki67 displaying cells were restricted mostly to the basal layer in the epidermis of non-lesional skin. Sections of the non-treated lesional skin showed, on the other hand, increased numbers of Ki-67 positive cells in the epidermis and numerous Ki-67 positive cells in the suprabasal layers of epidermis (FIG. 2H), as is well known for psoriasis. FIG. 2I shows return of both the frequency and the epidermal position of the Ki-67 antigen positive cells to normalcy in cyclopamine-treated lesional skin. Tissue sections of the junctional areas of the cyclopamine-treated lesional skin with the non-treated lesional skin (FIG. 2J) and with the non-lesional skin (FIG. 2K) show again the normalizing effect of cyclopamine on the frequency and epidermal position of Ki-67 positive cells and provide evidence of a concentration-dependent effect of cyclopamine.

The monoclonal antibody Ber-EP4 is known to label the basal layer cells in normal epidermis. The outer root sheath of hair follicles, where the hair stem cells are thought to reside, are also known to be labeled with Ber-EP4. FIG. 2L shows that non-lesional skin showed a normal pattern of labeling with Ber-EP4 (i.e. labeling of the basal layer cells). The non-treated psoriatic lesional epidermis, on the other hand, showed absence of labeling of the basal layer with Ber-EP4 under the same conditions (FIG. 2M). This Ber-EP4 detected abnormality of the basal layer cells in the psoriatic lesional epidermis, as far as we know previously undescribed, reverted to normalcy upon treatment of the lesion with cyclopamine (FIG. 2N).

FIG. 2O shows a Ber-EP4 stained tissue section from the cyclopamine-treated and non-treated junctional area of the psoriatic lesion. The normalizing action of cyclopamine on the psoriatic lesion at a distance suggests sensitivity of the Ber-EP4 detected abnormality to cyclopamine. Exposure of the basal cell carcinoma cells to cyclopamine was found earlier to cause their differentiation and caused loss of their Ber-EP4 staining (Taş S. et al, (2001) PCT/TR 01/00027). Basal cells of the normal epidermis exposed to cyclopamine under the same conditions, however, continued to be Ber-EP4 positive (Taş S. et al. (2001) PCT/TR 01/00027). FIG. 2P shows that basal cells of the non-lesional skin in psoriasis also remained Ber-EP4 positive after exposure to cyclopamine; the basal cell characteristics were maintained.

Cytokeratin 15, recognized by the C8/114B antibody, is found normally both in the hair follicle and in the basal layer cells in normal epidermis (Kanitakis J. et al (1999) Eur. J. Dermatol. 9:363-365). FIG. 2R shows the labeling of the basal layer cells by C8/144B in non-lesional skin. In contrast, basal layer cells of the non-treated psoriatic lesional skin were stained very weakly or not at all with the C8/144B antibody (FIG. 2S). On the other hand, epidermal basal layer cells in the cyclopamine-treated half of the lesional skin were normalized and became labeled by the C8/144B antibody (FIG. 2T). As both C8/144B and Ber-EP4 detect both the outer root sheath cells and the normal epidermal basal layer cells, the basal cell abnormality revealed by these two antibodies in the psoriatic lesional epidermis may be related or identical. Cyclopamine did not adversely affect the non-lesional skin and, similar to the situation with Ber-EP4, basal cells of the non-lesional epidermis that were exposed to cyclopamine continued to be positive for the cytokeratin 15 (FIG. 2U).

Infiltration of dermis with CD4 positive lymphocytes, a well-known feature of psoriatic plagues, was displayed by the non-treated psoriatic lesional skin (FIG. 2V). On the other hand, the CD4 positive lymphocyles infiltrating the psoriatic lesional skin were largely cleared from the cyclopamine-treated half of the lesional skin (FIG. 2W). Clearance of the CD4 positive lymphocytes from psoriatic lesional skin rapidly following application of cyclopamine thereto is highly surprising, since termination of the hedgehog/smoothened signalling was shown in the prior art to be necessary for the conversion of CD4-CD8 double negative T lymphocytes to the CD4-CD8 double positive T lymphocytes [Outram S V et al (2000) Immunity 13; 187-197]. Specifically, formation of CD4-CD8 double positive lymphocytes from CD4-CD8 double negative lymphocytes was shown to be enhanced by an anti Hh neutralizing monoclonal antibody [Outram S V et al (2000) Immunity 13:187-197]. Moreover, conversion of the CD4(−) and CD8(−) lymphocytes to the CD4 (+) and CD8(+) lymphocytes is viewed widely in the prior art as a requirement for their psoriatic plaque causing action [reviewed in Krueger J G (2002) Journal of the American Academy of Dermatology 46:1-23].

Genetic heterogeneity and different ages of the psoriatic patients as well as the localizational heterogeneity of psoriatic lesions throughout body invite evaluation of the use of cyclopamine on different patients and lesions. In this invention, treatments of unrelated patients ranging from 29 years of age to 57 years and treatments of psoriatic lesions localized on various body parts ranging from extremities to the trunk showed that cyclopamine was highly effective on every psoriatic lesion for which it was used and resulted in regression and clearance (7 separate lesions on different patients were treated at the time of writing of this invention) [with additional psoriatic patients and lesions treated since the filing of PCT/TR 02/00017, the number of separate lesions that have been treated exceeds 25 (summarized below)]. FIG. 1 D, FIG. 1 E and FIG. 1 F show that even when cyclopamine was applied for a day and then discontinued, the psoriatic plaque that received the treatment continued to regress and cleared totally. In this particular case the psoriatic plaque displayed decreased erythema on the 12th hour of treatment. Despite marked regression, it was still visible on the fourth day of follow-up. The lesion cleared after day 8 and the site of the treated lesion is displaying healthy-looking normal skin over a month of follow-up at the time of writing of this invention. Cyclopamine, applied topically on healthy skin as disclosed in this invention and earlier (Taş S. et al. (2001) PCT/TR 01/00027) had no detectable adverse effect. The longest duration of follow-up for a possible adverse effect of topical cyclopamine on healthy skin is over 14 months at the time of writing of this invention and no adverse effect has been found (skin sites of cyclopamine application were followed up also in the interval since the filing of PCT/TR 02/00017 and show healthy-looking normal skin and hair over a period of more than 31 months now suggesting functional preservation as well of the stem cells and long-term safety).

FIG. 3C to FIG. 3L show skin tissue sections from the non-lesional skin, non-treated psoriatic lesional skin and the cyclopamine-treated lesional skin of different patients (patients other than the one described in figures FIG. 2A to FIG. 2W) and further exemplify the uses and findings of this invention.

FIG. 3A, FIG. 3B and FIG. 3C show histopathological findings with non-lesional skin tissue, non-treated psoriatic lesional skin tissue and cyclopamine-treated psoriatic lesional skin tissue at the 24th hour of treatment and exemplify the cyclopamine-induced regression of the psoriatic plaque on a patient.

FIG. 3D, FIG. 3E and FIG. 3F show immunohistochemical staining for the Ki-67 antigen of non-lesional skin tissue, non-treated psoriatic lesional skin tissue and cyclopamine-treated lesional skin tissue at the 24th hour of treatment and exemplify the cyclopamine-induced regression of another psoriatic plaque.

FIG. 3G, FIG. 3H and FIG. 3I show immunohistochemical staining using the Ber-EP4 antibody of the non-lesional skin tissue, non-treated psoriatic lesional skin tissue and cyclopamine-treated psoriatic lesional skin tissue at the 24th hour of treatment and exemplify on another patient the Ber-EP4 detected abnormality of the basal layer cells in the psoriatic lesional skin as well as the cyclopamine-induced reversion to normalcy.

FIG. 3J, FIG. 3K and FIG. 3L show immunohistochemical staining with the C8/144B antibody of the non-lesional skin tissue, non-treated psoriatic lesional skin tissue and cyclopamine-treated psoriatic lesional skin tissue at the 24th hour of treatment and exemplify on a different patient the C8/144B detected abnormality of the basal layer cells in the psoriatic lesional skin as well as the cyclopamine-induced reversion to normalcy. Cytokeratin 15 that is bound by the C8/144B antibody [Kanitakis J. et al. (1999) European Journal of Dermatology 9:363-365] has been known as a protein expressed in the basal layer of epidermis and in the bulge region of the outer root sheath of hair follicles where the follicle/epiderm stem cells are thought to reside [Lyle S. et al. (1998) Journal of Cell Science 111:3179-3188]. As hedgehog/smoothened signaling is required for the maintenance of epithelial stem cells [Zhang Y. et al. (2001) Nature 410:599-604], consistent restoration of the lost cytokeratin 15 expression to the epidermal basal layer of psoriatic lesional skin following exposure to cyclopamine is surprising.

FIG. 4A shows a psoriatic plaque on the antecubital region of a 29-year old man prior to treatment. Approximately 20 μl of the cyclopamine cream (18 mM cyclopamine in the base cream described above) was applied onto this lesion every fourth hour for 24 hours. Treatment was then discontinued and the lesion was followed-up. The lesion showed decrease of erythema on the eighth hour of treatment and then continued regression also during the non-treated follow-up to reach to the state shown in FIG. 4B on day three and became undetectable on day four.

FIG. 4C shows another psoriatic plaque located on the deltoid region skin of the same patient prior to treatment. Approximately 20 μl of a cream preparation containing about 9 mM cyclopamine and about 0.55 mM clobetasol-17 propionate (in base cream) was applied onto this lesion every fourth hour. This lesion also displayed decrease of erythema on the eighth hour and became undetectable on day three (FIG. 4 D shows its appearance on the 68th hour). In the same patient other psoriatic lesions that were covered with a cream preparation containing about 1.1 mM clobetasol-17-propionate (but no cyclopamine) on every fourth or eight hour showed no detectable change during the same period (i.e. the lesions were persisting on day three). The enhanced therapeutic response to cyclopamine, even at half of the concentration we were using on other lesions of the same patient, prompted further evaluation of the therapeutic compositions comprising of cyclopamine and a corticosteroid. We evaluated in addition pretreatment of the psoriatic lesions with a topical corticosteroid followed by treatment with the cyclopamine cream (18 mM cyclopamine in base cream). FIG. 4E shows a psoriatic plaque on the hypochondrial region of the same patient prior to the applications of cyclopamine. This lesion was treated with the corticosteroid cream alone for 48 hours, corticosteroid was then discontinued and the treatment was switched to the applications of about 20 μl cyclopamine cream (18 mM cyclopamine in base cream) every fourth hour. FIG. 4F shows the lesion on the 24th hour of the cyclopamine cream applications and shows near complete disappearance of the lesion within only a day.

Severity of psoriatic lesions can be assessed on a semi-quantitative scale by giving separate scores for the erythema, elevation and scaling of a lesion and then by summing up the scores to obtain a score called the EES score of that lesion (Bowman P. H. et al. (2002) J. Am. Acad. Dermatol. 46:907-913). Table I shows comparisons of the therapeutic responses to various forms of treatment, evaluated by the EES scoring. It is seen that use of a therapeutic composition comprising of cyclopamine and a corticosteroid in the treatment of psoriatic lesions enhanced therapeutic effectiveness significantly in comparison to the use of a composition containing only cyclopamine in base cream. Pre-treatment of lesions with corticosteroid for a day, followed by treatment with the cyclopamine cream (18 mM cyclopamine in base cream), enhanced the therapeutic effectiveness similarly. Treatment of lesions with a topical corticosteroid (clobetasol 17-propionate, about 1.1 mM) alone, on the other hand, was unable to cause a significant regression or clearance of the lesions during the four days of treatment (Table I). Table I shows that psoriatic lesions treated for a day regressed but in general at a relatively slower pace in comparison to the continued use of cyclopamine (i.e. in comparison to the application every fourth hour of a therapeutic composition containing cyclopamine until the lesion cleared, usually within about 3-4 days). In the treatments for a single day, once again, a therapeutic composition comprising of cyclopamine and a corticosteroid or pre-treatment of lesion for a day with corticosteroid proved to be more effective than the use of cyclopamine alone (Table I). While the possibility of therapeutic effectiveness with a single day of treatment may be attractive to some patients, most patients are likely to prefer the faster clearance of lesions attained with the continued use of medication. Furthermore, some lesions, subjected to the single-day treatment (about 29%, all in the group treated with cyclopamine alone and none in the group treated with a composition comprising of cyclopamine and corticosteroid) failed to clear completely and exhibited even increase of the EES score around the end of 1st week (data not shown). Thus, uninterrupted use of a therapeutic composition comprising of cyclopamine and a corticosteroid offers to patients at present the fastest and most effective clearance of psoriatic lesions (Table I). The mechanism behind this synergistic action is not clear at present. However, lack of effectiveness of a corticosteroid alone during the approximately 2 to 4 days of treatment that suffices for the lesion-clearing action of a composition comprising of cyclopamine and a corticosteroid is consistent with the intervention by cyclopamine (but not by corticosteroids) with (a) key/proximal pathogenic event(s).

What might be the key/proximal pathogenic event(s) intervened by cyclopamine? While not wishing to be bound to any theory, we note the differentiation-inducing activity of cyclopamine as important in this regard. Studies published prior to this invention reported a blocking of the cellular differentiation by cyclopamine and suggested that cyclopamine may be used for preventing differentiation (Detmer K. et al (2000) Dev. Biol. 222:242; Berman D. M. et al. (2000) J. Urol. 153:240). However, we have found that the exposure of psoriatic lesional skin to cyclopamine induced rather differentiation of the epidermal cells. Re-appearance of the granular layer in the epidermis of the cyclopamine-treated psoriatic lesional skin shows that the block to differentiation in the psoriatic plaque was overcome by the cyclopamine treatment. Other findings shown in FIGS. 4G, 4H and 4J also show rapid induction of the differentiation of epidermal cells in the psoriatic lesional skin in response to the treatment with cyclopamine. Expression of the cell adhesion molecule CD44 is known to increase normally with the differentiation of epidermal basal cells to the upper layer spinous cells [Kooy A. J. et al (1999) Human Pathology 30:1328-1335] FIG. 4G shows a tissue section from the junctional area of non-treated psoriatic lesional skin (to the left of figure) and adjacent non-lesional skin (to the right of figure) with immunohistochemical staining for the CD44 antigen (anti-human CD44 antibody F10-44-2 binding to the CD44 standard was purchased from Novocastra Labs. Ltd., U.K. and immunohistochemical staining using peroxidase was performed under conditions recommended by the manufacturer). Both FIG. 4G and a closer-up view on FIG. 4H show decreased CD44 expression in the suprabasal and spinous layers in lesional epidermis in comparison to the situation in non-lesional epidermis. FIG. 4I, showing a tissue section from the lesional skin on the 24th hour of treatment with cyclopamine, shows rapid induction and normalization of the CD44 expression following treatment.

Epidermal growth factor receptor (EGFR) is another marker known to display downregulation of expression with the differentiation of epidermal keratinocytes. Expressions of both EGFR and one or more of its ligands are known to be markedly increased in psoriatic lesional epidermis and may set an autocrine stimulation loop. Ordinarily EGFR expression is not detected immunohistochemically in the suprabasal layers of normal epidermis and the return of EGFR expression to the basal layer of epidermis is regarded to be one of the first signs of effective treatment of psoriasis by various modalities [King L E Jr. et al (1990) Journal of Investigative Dermatology 95:10S-12S]. We evaluated the EGFR expression with anti-human EGFR antibody EGFR 113 (Novocastra Lab. Ltd., U.K.) and with immunohistochemical staining with peroxidase. FIG. 4J shows normal pattern of expression of the EGFR in non-lesional epidermis. Both placebo-treated and non-treated psoriatic lesional skin displayed marked overexpression of the EGFR in the suprabasal layers of epidermis (FIG. 4K). FIG. 4L shows that the EGFR expression was rapidly normalized within 24 hours of the treatment of psoriatic lesional skin with cyclopamine. Thus, treatment of psoriatic lesional skin with cyclopamine was accompanied with the rapid induction of differentiation and therapeutic effect by the criterion of EGFR expression.

FIG. 5A to FIG. 5E exemplify therapeutic results in another skin disorder known to be associated with inhibition of differentiation of epidermal cells in lesional skin following treatment as described above with patients having psoriasis vulgaris. Palmoplantar pustulosis (also known as pustulosis palmaris et plantaris) is a disease in which the skin of palms and soles shows chronic occurrences of pustules in patches of erythematous skin that show also scales, frequently on the hypothenar and thenar eminences of palms, lateral of soles and sides and back of heel. Skin other than in the palms and soles is typically spared and this is a clinical feature that can help to distinguish palmoplantar pustulosis from generalized pustular psoriasis and psoriasis vulgaris. Further distinguishing features of palmoplantar pustulosis are also known. For example the linkage of particular HLA alleles with psoriasis vulgaris and generalized pustular psoriasis known to affect the immune responses in these conditions could not be determined with patients having palmoplantar pustulosis (Karvonen J et al, Annals of Clinical Research 1975; 7:112-115). There are on the other hand also overlapping, shared features of palmoplantar pustulosis with these psoriasis forms and the coexistence of palmoplantar pustulosis with them in the same patients is observed in frequencies more than that would occur by chance, implying shared as well as different pathological processes in the occurrences of skin lesions in palmoplantar pustulosis and psoriasis vulgaris and generalized pustular psoriasis.

Conventional histopathological and other investigations of the skin biopsies from palmoplantar pustulosis patients have also shown shared pathological features of palmoplantar pustulosis with various forms of psoriasis besides one that is considered pathognomonic for palmoplantar pustulosis (e.g. Chopra A et al, Indian Journal of Dermatology, Venereology and Leprology 1997; 63:82-84). Occurrence of unilocular pustules in epidermis below the stratum corneum has been described as a diagnostic histopathological feature of palmoplantar pustulosis and the histopathological changes that have been mentioned under the “Background Of Invention” as typical histopathological signs of psoriatic skin lesions are also found in the lesional skin of palmoplantar pustulosis patients (Chopra et al, ibid). Parakeratosis determined in the epidermis of lesional skin of all palmoplantar pustulosis patients and nearly all psoriasis patients refers to the occurrence of nucleated cells in stratum corneum and is known to be a sign of inhibition of epidermal differentiation in the lesional skin of patients. The cell nucleus is lost during normal epidermal differentiation from the granular cells as they move to the stratum corneum and contribute to the formation of the normal epidermal barrier (e.g. Jensen J M et al, Journal of Investigative Dermatology 2000; 115:708-713 and references therein). Thus parakeratosis does not occur in normal skin.

FIG. 5A shows, prior to treatment, palm of a woman who had chronic erythematous skin lesions with scales and pustules restricted to her palms and soles. Physical examination and medical history did not reveal skin lesions elsewhere in her body. FIG. 5B shows her foot and sparing of the non-plantar skin by her disease. She had recurring sternocostoclavicular pain, sternal tenderness and further clinical signs of palmoplantar pustulosis as well and was a long time cigarette smoker. Various conventional treatments of psoriasis had been given to her with no or at best marginal improvement of her lesions and articular pain. She had stopped the treatments several weeks before the instant treatment. FIG. 5C shows her palm at the end of day 4 of treatment where a cream comprised of 9 mM cyclopamine and 0.55 mM clobetasol 17-propionate was administered to the lesional skin similar to that described above for psoriasis patients (about 25 μl cream was applied evenly to each cm² of lesional skin). The treatment is seen to have caused marked decrease of the erythema and scaling in the lesional skin and the pustules had become barely visible (FIG. 5C). The treatment was discontinued in this case on day 4 to allow the patient to return to her job in another city. The lesional skin was found to show improvement during the non-treated period following the 4-day treatment until about a month after the treatment. At that time the pustules continued to show disappearance but foci of erythema and scaling became visible in the former lesional skin region (FIG. 5D). A second treatment like in the previous 4-day treatment was repeated for about 40 hours during a two day period she was off work and rapid regression in lesion severity was again found already on day 2 of the second treatment. FIG. 5E shows the same palm about 10 days after this 40-hours treatment and shows marked improvement of lesional skin relative to the pre-treatment lesional skin (FIG. 5A) and also relative to that before the 40-hour treatment (FIG. 5D). Several areas of the former lesional skin are seen to have become clinically indistinguishable from non-lesional skin (i.e. to show disappearance of lesion) and no pustules are visible anywhere in the palm.

Administration of 0.55 mM clobetasol 17-propionate alone for 4 days as above to palmoplantar pustulosis lesions is known not to produce a perceptible effect and this patient had used that and other corticosteroids for longer periods without perceptible improvement. The instant drug treatment using a selective inhibitor of Hh/Smo signaling has been reproducibly effective in causation of rapid regression and disappearances of skin lesions within about a week of uninterrupted treatment with all patients treated so far who had different forms of psoriasis or another skin disorder in which the lesional skin shows inhibition of differentiation of the epidermal cells similar to the situation in psoriasis. The diversity of the genotypes of the investigated patients (they were all unrelated and most were born in different cities) suggest that their genetic predisposition to their condition was caused by different genes and/or by different alleles in view of the multiple different predisposing genes (the latter reviewed in Elder et al, ibid). Such genetic diversity along with the varying environmental risk factors in different individuals can be behind the varying periods from the initiation of treatment to achievement of lesion disappearance. The fact that the predisposing genes (and mostly also the environmental factors) would not be changed by the treatment can account for the reappearance of skin lesions after varying periods of disappearance. On the other hand a solution to the problem of patients is provided by the determination that the reappearing lesions can again be caused to disappear by a repeat of the described medicament administration that is effective for induction of differentiation of the epidermal cells in lesional skin and for that therapeutic result without a detectable safety shortcoming.

Psoriasis and palmoplantar pustulosis are known to cause lesions not only in skin but also in other tissues and organs that include the joints and bones. Both in psoriasis and palmoplantar psoriasis, occurrence of arthritis is found to be associated with the involvement of nails of patients (Green L et al, Annals of Rheumatic Diseases 1981; 40:366-369; Burden A D et al, British Journal of Dermatology 1996; 134:1079-1082). Involvement of nails is considered a readily recognized predictor of joint involvement. The pathology and clinical findings concerning the involvement of nails in psoriasis and palmoplantar pustulosis, while having similarities, show also differences that help in their differential diagnosis (Burden et al, ibid). FIG. 6A shows an involved nail of the palmoplantar pustulosis patient who had also joint involvement and whose palm and sole lesions are shown in FIG. 5A, FIG. 5B. Nails were treated by topical administration of a cream comprised of cyclopamine every fourth hour for four days to the nail bed and about 0.5 cm band of surrounding skin similar to the administrations described above in treatments of psoriasis patients. The skin around the nail bed, which was erythematous prior to treatment (FIG. 6A), showed marked improvement by day 4 of treatment (FIG. 6B). FIG. 6C shows causation of disappearance of the nail dystrophy by the described administrations of a medicament comprised of a selective inhibitor of Hh/Smo signaling and shows causation of normal healthy nail growth that is visible in the proximal of nail. Other nail treatments like in this example have also provided similar disappearance of the nail dystrophy.

We disclose that besides in psoriasis, cyclopamine is effective in inducing differentiation of epidermal cells also in other skin diseases having a unifying feature of being associated with the inhibition of cellular differentiation. The treatment we describe can therefore provide good therapeutic effectiveness on those diseases associated with impairment of cellular differentiation.

Above-summarized immunohistochemical findings as well as the re-appearance of granular layer in the epidermis of cyclopamine-treated psoriatic lesional skin point out to the rapid overcoming of the differentiation block of epidermal cells following the described treatment. While not wishing to be bound by any particular theory, the finding of this invention on the Ki-67 antigen expression by the epidermal cells in the cyclopamine-treated psoriatic lesional skin may also be related to an induction of differentiation by cyclopamine. Disappearance of proliferating cells from the suprabasal layers of the psoriatic lesional epidermis following exposure to cyclopamine (FIG. 2H versus FIG. 2I and FIG. 3E versus FIG. 3F) may be consequential to a regain of the potential for terminal differentiation. In this regard, it should also be noted that a highly potent inhibitor of cellular proliferation, methotrexate, has for many years been used in the treatment of psoriasis but display much lesser (and delayed) therapeutic effectiveness than the treatment described herein. Nevertheless, regardless of the precise mechanism, return of the overproliferative activity of the psoriatic lesional epidermis to normal levels following exposure to cyclopamine is by itself beneficial (has therapeutic value).

Rapid clearance of the psoriatic plaques as described in this invention (as fast as within a day) can be contrasted with the average of 6 to 8 weeks of treatment required for the conventional treatments to become effective (Al-Suwaidan S. N. et al. (2000) J. Am. Acad. Dermatol. 42:796-802; Lebwohl M. et al. (2001) J. Am. Acad. Dermatol. 45:649-661). Thus, the treatment described in this invention represents a major improvement and solution to a long-standing problem. The rapidity of the response to cyclopamine suggests in addition intervention with a proximal causative event involved in the formation of the psoriatic plaque.

As there is evidence for the involvement of the hedgehog/smoothened signal transduction pathway in the maintenance of epidermal stem cells, untoward side effects of cyclopamine on skin are, a priori, possible and must be excluded. As described in this invention and earlier (Taş S. et al. (2001) PCT/TR 01/00027), under the described concentration and dosing conditions no adverse effect has been detected. Lack of detectable side effects of the described treatment, combined with hitherto unachieved high topical effectivity represents a solution to the therapeutic dilemma that aggressive uses of conventional treatments often result in unacceptable adverse effects but their less aggressive uses may leave the patient with his/her lesions of psoriasis (AI-Suwaidan S. N. et al (2000) J. Am. Acad. Dermatol. 42:796-802).

TABLE I
Semi-quantitative Evaluation Of The Responses Of Psoriatic
Lesions To Different Types Of Treatment

| | ESS score* of the psoriatic | | |
|---|---|---|---|
| | lesions before and on days 2 | | |
| | and 4 of treatment (mean ± S.D.) | | |
| Treatment | Pre-Treatment | Day 2 | Day 4 |
| Base Cream | 6.6 ± 1.0 | 6.2 ± 0.6 | 6.0 ± 0.6 |
| CS Cream | 6.8 ± 0.6 | 6.4 ± 0.8 | 6.5 ± 0.8 |
| Cyclopamine Cream | 6.6 ± 0.4 | 1.4 ± 1.0 | 0.6 ± 0.5 |
| (Cyclopamine + CS) Cream | 6.7 ± 0.9 | 0.5 ± 0 | 0 ± 0 |
| Cyclopamine Cream, 1 day | 6.8 ± 1.6 | 1.8 ± 0.6 | 1.1 ± 0.5 |
| (Cyclopamine + CS) Cream, | 6.7 ± 1.3 | 1.1 ± 0.4 | 0.4 ± 0.3 |
| 1 day | | | |

*Sum of the erythema, elevation and scaling scores (each on a 0 to 4 scale with 0.5 increments). Numbers of lesions scored in each treatment category are 5, 4, 5, 5, 7 and 7 (respectively, in the same order as shown in the Table starting with base cream). Scores of the lesions that were excised before day 4 for histopathological/immunohistochemical analyses are not included in this evaluation and calculations.
“CS Cream” refers to treatment with a cream preparation containing about 1.1 mM clobetasol-17-propionate.
“Cyclopamine Cream” refers to treatment with 18 mM cyclopamine in the base cream.
“(Cyclopamine + CS) Cream” refers to treatment with a cream preparation containing about 9 mM cyclopamine and about 0.55 mM clobetasol-17-propionate or to treatment with a cream preparation containing 18 mM cyclopamine after a day of pre-treatment with a cream preparation containing about 1.1 mM clobetasol-17-propionate. Results of these two types of treatment were similar and were therefore calculated as a single group.
The “1 day” treatments refer to treatments where treatment with a cream containing cyclopamine was continued for one day only. The lesions were then followed up without treatment and the EES scores were determined.

1. A method of treating a human subject having a skin disorder presenting non-tumoral skin lesions comprising:
determining if the skin disorder in said subject is a skin disorder that shows inhibition of differentiation of epidermal cells in the skin lesions; and if it is identified as such a disorder,
administering to the subject a medicament comprising cyclopamine or another molecule that, like cyclopamine, is a molecule that selectively inhibits Hedgehog/Smoothened signaling,
wherein said administering of cyclopamine or said another molecule is in a dose that is sufficient to induce differentiation of the epidermal cells in lesional skin and causes regression or disappearance of the skin lesions.

2. A method as in claim 1, wherein said another molecule binds to the Smoothened protein to inhibit Hedgehog/Smoothened signaling.

3. A method as in claim 1, wherein said another molecule is a functionally equivalent derivative of cyclopamine.

4. A method as in claim 1, wherein said another molecule is combined with a corticosteroid in said medicament or the skin lesions are pre-treated with a corticosteroid and said combining or pre-treatment provides increased therapeutic effectiveness and causes faster regression or disappearance of the skin lesions.

5. A method as in claim 1, wherein said skin disorder is determined to be psoriasis.

6. A method as in claim 1, wherein said medicament is in the form of a cream or ointment or gel or hydrogel or foam or another form formulated for topical administration or formulated for controlled release or for systemic administration or cyclopamine or said another molecule is entrapped in liposomes or adsorbed onto a dermal patch.

7. A method of treating psoriasis, comprising administration of a medicament comprising cyclopamine or another molecule that binds to the Smoothened protein to inhibit Hedgehog/Smoothened signaling,
wherein said medicament is administered to a psoriasis patient in a sufficient dose that provides, within eight days of administration, an average of 80% or greater decrease in the severity of psoriatic lesions, taking into account the elevation from skin surface, erythema and scaling of lesions.

8. A method as in claim 7, wherein said medicament is in the form of a cream or ointment or gel or hydrogel or foam or another form formulated for topical administration or formulated for controlled release or for systemic administration or cyclopamine or said another molecule is entrapped in liposomes or adsorbed onto a dermal patch.

9. A medicament for treatment of psoriasis or another non-tumoral skin disease that shows inhibition of differentiation of the epidermal cells in skin lesions,
characterized in that the medicament comprises cyclopamine or a another molecule that, like cyclopamine, is a molecule that selectively inhibits Hedgehog/Smoothened signaling,
wherein the medicament is a pharmaceutical formulation having cyclopamine or said another molecule in a quantity effective for causation of differentiation of the epidermal cells in lesional skin and regression or disappearance of the skin lesions, and
administered in a dose effective for said differentiation and regression or disappearance.

10. A medicament as in claim 9, wherein said another molecule binds to the Smoothened protein to inhibit Hedgehog/Smoothened signaling.

11. A medicament as in claim 9, wherein said another molecule is a functionally equivalent derivative of cyclopamine.

12. A medicament as in claim 9, wherein the medicament is in the form of a cream or ointment or gel or hydrogel or foam or another form formulated for topical administration or formulated for controlled release or for systemic administration or cyclopamine or said another molecule is entrapped in liposomes or adsorbed onto a dermal patch.
